(19) United States
(12) Patent Application Publication
(10) Pub. No.: US 2016/0183962 A1
(43) Pub. Date: Jun. 30, 2016

(54) Apparatus And Methods For Treating Undesired Viens

(71) Applicant: Greg Spitz, Batavia, IL (US)

(72) Inventor: Greg Spitz, Batavia, IL (US)

(21) Appl. No.: 14/972,485

(22) Filed: Dec. 17, 2015

Related U.S. Application Data
(60) Provisional application No. 62/093,185, filed on Dec. 17, 2014.

(51) Int. Cl. A61B 17/32; A61B 1/05; A61B 1/00; A61B 17/3205; A61B 1/06

(52) U.S. Cl. CPC A61B 17/32002; A61B 17/3205; A61B 1/0684; A61B 1/00009; A61B 1/05; A61B 1/00032; A61B 2017/320028; A61B 2017/00778

(57) ABSTRACT

A surgical apparatus removing veins in a venous system of a patient includes an elongated shaft having a first end and a second end. The second end has a cutting edge disposed on a distal edge of an aperture. A reciprocating member is disposed within the elongated shaft. A second end of the reciprocating member includes a cutting member to contact against the cutting edge of the aperture and the reciprocating member be reciprocated in response to electrical energy.

### FIELD OF THE INVENTION

The present invention relates generally to surgical apparatus and procedures. More particularly, it relates to methods and apparatus for treatment of veins, such as, varicose and spider veins, in a venous system of a patient.

### BACKGROUND

Ten to fifteen percent of the population is plagued with varicose veins and spider veins. These veins have usually lost their ability to carry blood back to the heart and blood often accumulates in these veins. As a result, the veins may become swollen, distorted, and prominent. This condition is usually caused by inefficient or defective one-way valves which prevent blood from draining back through the vein.

A number of factors can contribute to the development of varicose veins and spider veins, including heredity, obesity, posture, standing or sitting for long periods of time, hormonal shifts, and excessive heat. Varicose veins may cause patients to experience various symptoms, such as, for example, aching, burning, swelling, cramping, and itching, while more serious complications of varicose veins can include thrombophlebitis, dermatitis, hemorrhage and ulcers. If these varicose veins are not treated, blood clots may form in the vein and phlebitis or inflammation of the inside lining of the vein may occur. Even absent such symptoms, many patients seek medical treatment of varicose or spider veins for cosmetic reasons.

Various approaches have been developed to treat varicose veins. In less complicated cases, elevation of the legs and use of support hosiery may be sufficient therapy to stop or slow the progression of the varicose veins. Alternatively, a technique called “sclerotherapy” may be used to treat varicose veins. In this procedure, the affected veins are injected with a sclerosing solution, such as sodium tetradecyl sulfate or pilocainol. Approximately one injection of the solution is usually administered for every inch of the affected veins and multiple injections may be administered during a treatment session. The sclerosing solution causes subsequent inflammation and sclerosis of the veins. The sclerosis results in localized scarring or closure of the veins, which forces re-routing of the blood away from the affected veins.

However, patients usually have to undergo two or more sclerotherapy treatments in order to alleviate the varicose and spider veins to a satisfactory degree. Other fine reddish blood vessels may also appear around the treated area, requiring further injections. Nevertheless, the sclerotherapy technique may not be a permanent or complete solution since the condition of the varicose and spider veins may re-occur within five years.

Sclerotherapy also has other potential complications, including browning splotches or bruising of the skin, formation of blood clots in the veins, inflammation, adverse allergic reactions, ulceration, phlebitis, anaphylactic overdose, ischemia, skin or fat necrosis, and peripheral neuropathy. Furthermore, sclerotherapy cannot be applied to the saphenous vein in the upper thigh region due to the risk of sclerosis of deep veins. Thus, the sclerotherapy technique is often combined with an operative procedure, such as ligation of a portion of the saphenous vein.

Another technique to treat varicose veins is called stabavulsion phlebectomy with hooks. In this technique, one or more incisions are made in the skin of the patient, and a hook is inserted into the incision to grip or hook the veins to be removed. When the veins are grabbed, the veins are pulled through the surgical incision and severed. However, this procedure usually requires two surgeons to perform the procedure and takes about 2-3 hours. In addition, this procedure usually requires multiple incisions in the patient in order to hook the affected veins. Furthermore, it is often difficult to completely remove the entire affected veins using this procedure.

Varicose veins can also be removed by a procedure commonly referred to as “stripping.” To remove a saphenous vein using this stripping procedure, a large incision is made near the groin area of a patient and the saphenous vein is separated from the femoral vein. Multiple large incisions are made along the leg in order to sever and ligate the tributary veins of the saphenous vein. A vein stripper, such as a wire, is then inserted into the lumen of the saphenous vein. The wire is then inserted through the lumen of the saphenous vein and tied to the lower end of the vein. The wire is then removed through the groin incision to extract the vein. After the saphenous vein is completely removed from the leg, the incisions along the leg are closed.

However, the stripping procedure is usually painful and often requires overnight hospitalization. In addition, large numerous incisions are usually required to remove the saphenous vein and its branches which often leave permanent unsightly scars along the leg of a patient. Additionally, the large incisions create a risk of infection to the patient and may not heal properly, especially patients who have poor circulation in their extremities. There are also associated complications with this technique, such as, for example, blood loss, pain, infection, hematoma, nerve injury, and swelling.

### SUMMARY OF THE INVENTION

In view of the above, the present invention provides methods and apparatus for treating undesired veins, such as, varicose and spider veins, in a venous system of a patient. The methods and apparatus provide improved techniques for treating spider and varicose veins. One device for removing veins in a venous system of a patient includes an elongated shaft having a first end and a second end. The second end of elongated shaft has an aperture. A cutting edge is disposed on the distal edge of the aperture. A reciprocating member is disposed within the elongated shaft and having a first end and a second end. The second end of the reciprocating member has a cutting member to contact against the cutting edge of the aperture. The reciprocating member being reciprocated in response to electrical energy.

One method of treating undesired veins in accordance with the present invention includes the steps of making at least one incision through the skin layer of a patient in close proximity the undesired veins, inserting a distal end of a surgical instrument having a cutting element, and advancing the cutting element end of the surgical instrument to a position adjacent to a selected vein. The method also includes the steps of energizing surgical instrument to cause the cutting element to reciprocate at a desired speed, contacting the cutting element against the undesired vein while the cutting element is moving to break up the vein into particles, and withdrawing the distal end of the surgical instrument and closing the at least one small incision.

The invention, together with further attendant advantages, will best be understood by reference to the following detailed description of the presently preferred embodiments of the invention, taken in conjunction with the accompanying drawings. The drawings have not been drawn to scale. It is to be understood that both the foregoing general description and the following detailed description are exemplary and explanatory and are intended to provide further explanation of the invention as claimed.

### BRIEF DESCRIPTION OF THE DRAWINGS

FIG. 1 is a side elevational view of a surgical system to remove undesired veins in a venous system or a patient;

FIG. 1A is a fragmentary side view of the distal end of a surgical instrument of the surgical system of FIG. 1;

FIG. 2 is a cross-sectional view taken about line 2-2 of FIG. 1;

FIG. 3 is a diagrammatical view of undesired veins being removed from the legs of a patient by the surgical system of FIG. 1;

FIG. 4 shows a pattern of movement of the surgical instrument of the surgical system of FIG. 3 to remove undesired veins;

FIG. 5 is a cross-sectional view taken about line 5-5 of FIG. 4;

FIG. 6 is a side elevational view of another embodiment of a surgical instrument to remove undesired veins in a venous system or a patient;

FIG. 7 is a side elevational view of another embodiment of a distal end of a surgical instrument to remove undesired veins in a venous system or a patient;

FIG. 7A is a fragmentary side view of the distal end of a surgical instrument of FIG. 7;

FIG. 8 is a side elevational view of another embodiment of a surgical system to remove undesired veins in a venous system or a patient;

FIG. 9 is a side elevational view of another embodiment of a surgical instrument to remove undesired veins in a venous system or a patient;

FIG. 10 is a side elevational view of another embodiment of a surgical instrument to remove undesired veins in a venous system or a patient;

FIG. 11 is a side elevational view of another embodiment of a surgical instrument to remove undesired veins in a venous system or a patient;

FIG. 12 is a side elevational view of another embodiment of a surgical instrument to remove undesired veins in a venous system or a patient; and

FIG. 13 is a side elevational view of another embodiment of a surgical instrument to remove undesired veins in a venous system or a patient.

### DETAILED DESCRIPTION OF THE PRESENTLY PREFERRED EMBODIMENTS

Before explaining the preferred embodiment in detail, it should be noted that the invention is not limited in its application or use to the details of construction and arrangement of parts illustrated in the accompanying drawings and description, because the illustrative embodiment of the invention may be implemented or incorporated in other embodiments, variations and modifications, and may be practiced or carried out in various ways. Furthermore, unless otherwise indicated, the terms and expressions employed herein have been chosen for the purpose of describing the preferred embodiment of the present invention for the convenience of the reader and are not for the purpose of limitation.

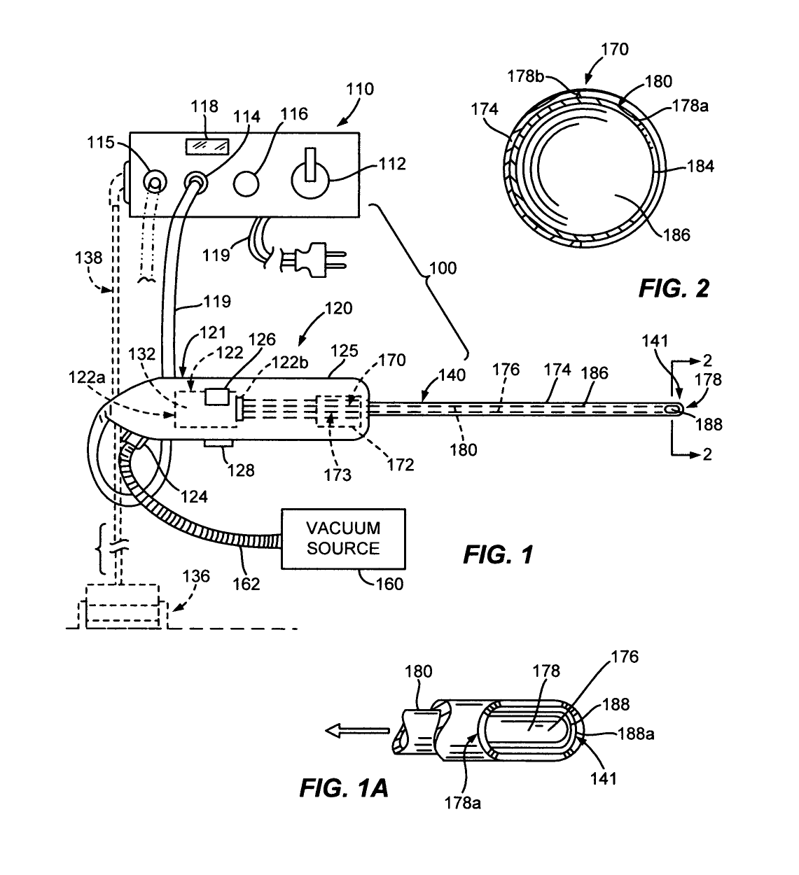

Referring now to the drawings in detail, and particularly to FIG. 1, a preferred embodiment of a surgical system 100 to remove undesired veins in a venous system of a patient is illustrated. The surgical system 100 allows a single surgeon to remove veins from a patient more quickly than traditional techniques. The undesired veins can be completely removed with minimal scarring and without hospitalization. The surgical system 100 can also allow two or more surgeons to remove undesired veins of a patient at the same time.

As shown in FIG. 1, the surgical system 100 generally includes a control unit 110 and a surgical instrument 120. The control unit 110 sends electrical signals through a cable 119 at a selected amplitude and frequency. The electrical signals energize a motor 122 of the surgical instrument 120 which reciprocates a cutting element 141 at a selected speed. The speed of the cutting element 141 of the surgical instrument 120 is controlled and monitored by the control unit 110.

The surgical instrument 120 of the surgical system 100 can be utilized by a surgeon to remove undesired veins in a patient. A second handpiece assembly (not shown) may also be connected to the control unit 110 to allow a second surgeon to remove undesired veins in another portion of the patient. It will be recognized that any suitable number of surgical instruments 120 can be attached to the control unit 110 to remove undesired veins.

Referring again to FIG. 1, the control unit 110 of the surgical system 100 preferably includes a control system (not shown) integral to the control unit 110, an on-off switch 112, jacks or connectors 114 and 115, a power control switch 116, and a display 118. The control unit 110 sends signals to the surgical instrument 120 to control the acceleration, deceleration, and speed of the cutting element 141 of the surgical instrument 120. The control unit 110 may also monitor the speed of the cutting element 141 in order to limit the speed and the force of the cutting element 141 to within preselected limits for optimum performance of the surgical instrument 120 being used. It will be recognized that the control unit 110 may drive the cutting element 141 at any suitable speed and force.

The on-off switch 112 of the surgical system 100 controls the electrical power to the control unit 110 to allow the surgeon to actuate the cutting element 141 of the surgical instrument 120. When the on-off switch 112 is activated, the control unit 110 provides power to drive the cutting element 141 of the surgical instrument 120 at a desired speed.

The proximal end of the cable 112 is attached to jack 114 of the control unit 110. Although only two jacks 114 and 115 are shown, it is contemplated that any suitable number of jacks may be incorporated into the control unit 110 to allow other surgical instruments to be coupled to the control unit 110. The control unit 110 also has a power line 118 for insertion in an electro-signal unit or conventional outlet. It is contemplated that the control unit 110 may also be powered by a direct current (DC) source, such as a battery.

Referring still to FIG. 1, the surgical instrument 120 generally includes a handpiece assembly 121 and a blade assembly 140. The handpiece assembly 121 includes a motor 122, a suction nozzle 124, housing or an outer casing 125, a trigger mechanism or power switch 126, and a vacuum switch 128. The distal end of the handpiece assembly 121 is sized and configured to receive the blade assembly 140, and the proximal end of the handpiece assembly 121 is coupled to the control unit 110 by the cable 119. The cable 119 may include ducts or vents to allow air or a cooling fluid to be introduced into the handpiece assembly 121 to cool the motor 122.

The housing 125 of the handpiece assembly 121 is adapted to isolate the operator from the inner components of the handpiece assembly 121. The housing 125 is preferably substantially cylindrically shaped and is adapted to be held by a user in a conventional manner, but may be any suitable shape or size which allows it to be grasped by the user. While a single or unitary component housing 125 is illustrated, the housing 125 may comprise multiple parts or pieces.

The housing 125 of the handpiece assembly 121 can be constructed from a plastic, titanium, aluminum, or steel. It is also contemplated that the housing 125 may be made from a variety of materials including other plastics (i.e., liquid crystal polymer (LCP), nylon, or polycarbonate) or any other suitable material.

The triggering mechanism 126 of the handpiece assembly 121 allows a surgeon to activate the control unit 110 so that power may be continuously supplied to the motor 122 of the handpiece assembly 121. The triggering mechanism 126 preferably includes a switch incorporated in the handpiece assembly 121. It will be recognized that the triggering mechanism 126 may alternatively include a foot activating switch 136 that is detachably coupled or attached to the control unit 110 by a cable or cord 138.

The suction nozzle 124 of the handpiece assembly 121 is coupled to the vacuum source 160 by a hose 162. When the vacuum source 160 is activated, the vacuum source 160 creates suction or a vacuum through a conduit (not shown) defined through the handpiece assembly 121 and the blade assembly 140. The vacuum source 160 causes vein tissue and other fluids to be drawn from the surgical site through the conduit of the blade assembly 140 and handpiece assembly 121 into the vacuum source 160. The vacuum source 160 can be any suitable vacuum or suction source to remove vein tissue from the surgical site. It is also contemplated that the vacuum source 160 can be a separate tool, such as, for example, a cannula, or any other suitable instrument that can be inserted in the incision to remove vein tissue.

The motor 122 of the handpiece assembly 121 is preferably disposed within the handpiece assembly 121. The motor 122 receives power from the control unit 110 over wires (not shown) in the cable 112. The motor 122 preferably includes a motor housing 122a and a drive shaft 122b. When the motor 122 is activated, the drive shaft 122b reciprocates the cutting element 141 of the surgical instrument 120 back and forth at a desired speed. It will be recognized that the motor 122 can be any suitable device that can drive or actuate the cutting element 141 of the surgical instrument 120. The blade assembly 140 can also be rotated clockwise or counter-clockwise in a ratchet-like manner. This allows a surgeon to rotate or orient the blade assembly 140 with respect to the handpiece assembly 121 allowing the cutting member 141 to be positioned in a desired orientation.

The blade assembly 140 of the surgical instrument 120 preferably includes an elongated member 170 and a reciprocating member 180. The blade assembly 140 is preferably a disposable unit to eliminate re-sterilization of the portions of the assembly that enters into the patient's body. Therefore, the blade assembly 140 is removed from the handpiece assembly 121 after use and disposed of.

The elongated member 170 of the blade assembly 140 preferably includes an outer stationary member 174, a conduit 176 extending longitudinally therethrough and one or more ports or openings 178 at its distal end (one being shown). The distal end of the elongated member 170 is sized for insertion into an incision of the body via a puncture opening made through the skin. The outer stationary member 174 of the elongated member 170 preferably has a length of about 5 inches and is fabricated from stainless steel. It is contemplated that the elongated member 174 may have any suitable length and can be manufactured from any suitable material. It is contemplated that the distal end of the elongated member 170 may be curved at a preselected angle or may be articulated or rotated in various directions as shown in FIG. 6. The curved blade assembly allows a surgeon to manipulate the distal end of the blade assembly to a desired position to remove veins.

The port 178 of the elongated member 170 is preferably located at or near the distal end of the elongated member 170. As shown in FIGS. 1A and 2, the port 178 preferably has a generally rectangular or elliptical shape and includes a cutting edge 178a. The port 178 allows vein tissue to enter into the aperture 176 so that the cutting element 141 of the surgical instrument 120 can sever the tissue as further described below. It will be recognized that the port 178 can be any size and shape to receive vein tissue. It is also contemplated that a guard (not shown) may also be disposed across the port 178 to limit the amount of extraneous tissue that can enter into the port 178 to be severed.

As shown in FIG. 1, the reciprocating member 180 of the blade assembly 140 preferably includes an aperture 176 extending longitudinally therethrough and one or more ports or openings 188 (one being shown) disposed near or at its distal end. The reciprocating member 180 is preferably disposed coaxially within the outer stationary member 172 of the blade assembly 140. The proximal end of the reciprocating member 180 is coupled to the drive shaft 122b of the motor 122. When the drive shaft 122b of the motor 122 is energized, the drive shaft 122b reciprocates the reciprocating member 180 at a desired speed. The reciprocating member 180 can be reciprocated at various speeds by the motor 122. The reciprocating member 180 is preferably fabricated from stainless steel. It is contemplated that the reciprocating member 180 may be constructed from any suitable material.

As shown in FIG. 1A, the port 188 of the reciprocating member 180 has a generally cylindrical shape and preferably includes a cutting edge 188a. The cutting edge 188a of the reciprocating member 180 is sharp and cooperates with sharpened edge 178a of the port 178 to sever and cut vein tissue. Vein tissue is cut into small pieces when the cutting edge 188a of the reciprocating member 180 is retracted against the edge 178a of the port as further described below. The cut pieces of the vein are drawn through the aperture 176 of the reciprocating member 180 and through the suction nozzle 124 of the handpiece assembly 121 by the suction applied by the vacuum source 160. The cutting element 141 may also comprise a blade, abrading burr, or ultrasonic element.

FIG. 1A shows the reciprocating member 180 of the blade assembly 140 in its extended position so that an opening is created between the cutting edge 188a of the reciprocating member 180 and the cutting edge 178a of the outer stationary member 180. When the cutting edge 188a of the reciprocating member is retracted toward the cutting edge 178a of the outer stationary member 80, the vein tissue within the opening is severed or sheared between the cutting edge 188a of the reciprocating member 180 and the cutting edge 178a of the elongated member 170. The severed vein tissue is drawn through the conduit of the elongated member and through the suction nozzle 124 of the handpiece assembly 121 by the suction applied by the vacuum source 160. It is also contemplated that the cutting element 141 may include any suitable cutting member and may be vibrated at a high rate of speed (i.e., in an ultrasonic range).

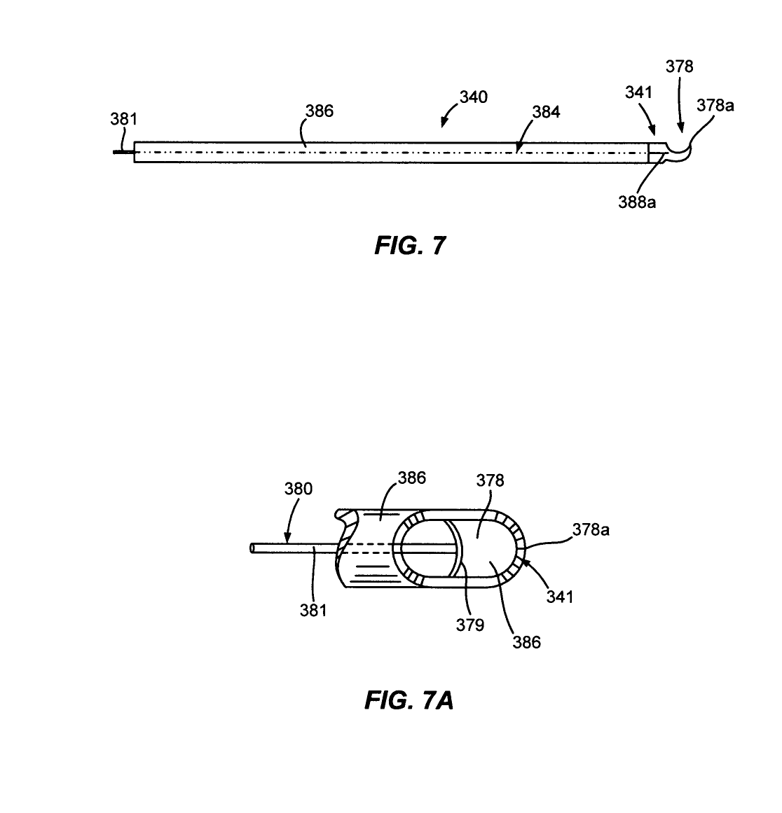

As shown in FIGS. 7 and 7a, another embodiment of a blade assembly 340 that can be attached to the surgical instrument 120 is illustrated which in many respects corresponds in construction and function to the previously described blade assembly 140 of FIG. 1. Components of the blade assembly 340 which generally correspond to those components of the blade assembly 140 of FIG. 1 are designated by like reference numbers in the three-hundred series.

The blade assembly 340 preferably includes a reciprocating member 384 and an outer stationary member 386. The outer stationary member 386 preferably includes a conduit extending longitudinally therethrough one or more ports or openings 378 at its distal end (one being shown). The port 378 has a generally rectangular or elliptical shape and preferably a cutting edge 378a at its distal end.

The reciprocating member 380 of the blade assembly 340 preferably includes a shaft 381 and a cutting element 379. The shaft is preferably disposed coaxially within the outer stationary member 386. The shaft 381 preferably has a circular cross-section shape and is preferably fabricated from stainless steel. It is contemplated that the shaft 381 may be constructed from any suitable material.

The cutting element 379 of the reciprocating member 380 is disposed at the distal end of the shaft 381. The cutting element 379 is configured for cutting vein tissue when the cutting member 379 is reciprocated back and forth. When the shaft 381 is reciprocated, the cutting element 379 is extended against the cutting edge 378a. The vein is cut by the cutting element 379 and cutting edge 388a into small pieces. The pieces are drawn through the aperture of the outer stationary member 384 and through the suction nozzle of the handpiece assembly by the suction applied by the vacuum source. It is also contemplated that the cutting element 379 may include any suitable cutting implement and may be vibrated at a high rate of speed (i.e., in an ultrasonic range).

Referring now to FIGS. 1-5, the operation of the surgical system to remove undesired veins, such as, varicose and spider veins, of legs of a patient will be described. Initially, a lower extremity venous doppler is taken of the patient to reveal the incompetent valves of the veins between the superficial vein system and the deeper vein system. If there is incompetence in the greater saphenous veins going into the femoral veins and the lesser saphenous veins going into the popliteal veins, the greater saphenous veins will be ligated and divided. During this procedure, the patient may undergo a general anesthetic, regional anesthetic (i.e., spinal or epidural), or a local anesthetic.

To disconnect the saphenous vein, an incision is made in the groin area to ligate the saphenous vein at its juncture with the femoral vein. All branches of the saphenous vein are ligated and divided with titanium clips and the main greater saphenous vein is ligated proximally and distally. Alternatively, the saphenous vein can be disconnected from the deep veins at a lower point along the leg, such as behind the knee at the lesser saphenous-popliteal junction. This alternative technique may be advisable in circumstances in which treatment of various veins is only necessary in the lower leg.

When the saphenous vein is disconnected from the femoral vein, the blood from the deep veins will be prevented from flowing backing into the saphenous vein, eliminating the primary cause of the varicose veins. While blood can still enter the saphenous vein through the numerous tributary veins, the subsequent permanent closure of the saphenous vein will effectively prevent this occurrence. Once the saphenous vein is disconnected from the femoral vein, the varicose and/or spider veins can be treated. It will also be recognized that the saphenous vein may not have to be disconnected.

To remove the undesired veins, the patient is placed in a trendelenburg position, i.e., feet up, and the incision site is prepared. After proper preparation of the incision site, a surgeon makes a small incision 152 through the skin layer and subcutaneous tissue of the patient. The incision is approximately 2-3 mm and only needs to be large enough to permit the distal end of the surgical instrument 120 to pass therethrough. The incision 152 may be made by a blade, such as a small surgical scalpel, such as a Number 67 scalpel blade. The incision 152 is preferably made near the center of the undesired veins to reach the most veins. It should be apparent that the selection of the incision 152 is for exemplary purposes only and the incision may be made at any suitable location.

After completing the incision 152, the distal end of the surgical instrument 120 is inserted through the incision 152 made at the skin with the motor 122 of the handpiece assembly 121. It is also contemplated that the distal end of the surgical instrument 120 may have a sharp point that enables it to be inserted through the skin layer without having to first make an incision. For example, the surgical instrument 120 may have a retractable scalpel blade.

Once the distal end of the surgical instrument 120 is passed through the incision, the surgeon then activates the switch 128 to cause vacuum suction. The distal end of the surgical instrument 120 is placed on the undesired vein in order to move the vein away from the skin and to remove as much vein tissue with the vacuum suction. The distal end of the surgical instrument 120 is then guided underneath or on the side of the vein to be removed. The surgeon then activates the switch 126 to actuate the cutting element 141 of the surgical instrument 120 at a predetermined speed. The surgeon can control reciprocation speed using the switch 126 on the handpiece assembly 121 or the foot switch 136. The cutting element 141 can preferably reciprocate over a wide range of speeds. Saline can also be introduced into the surgical site.

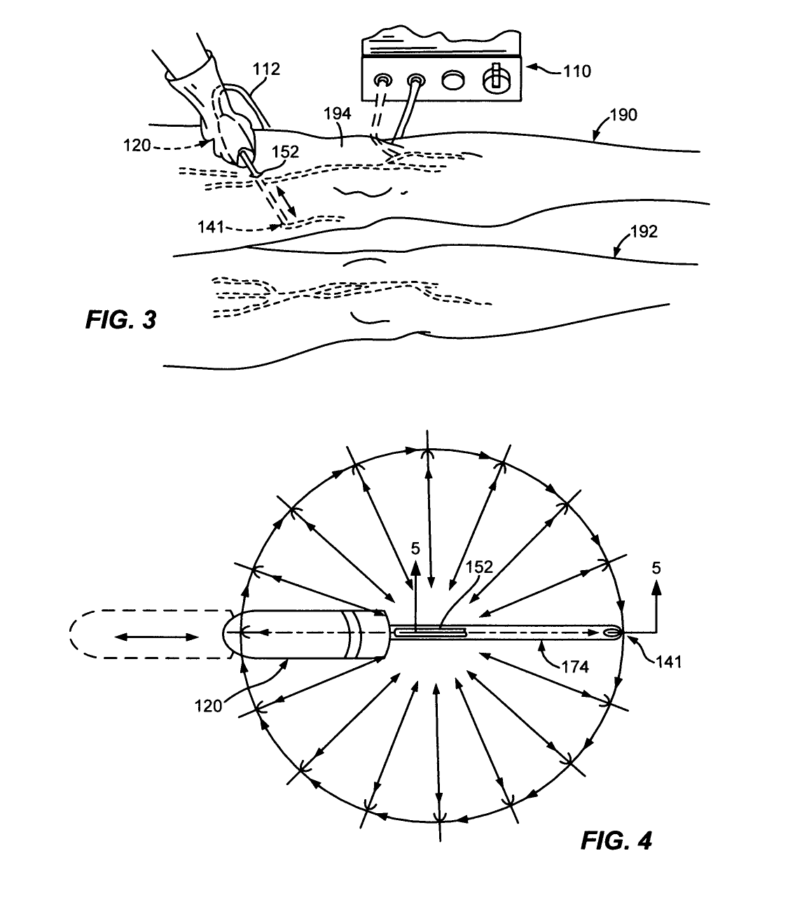

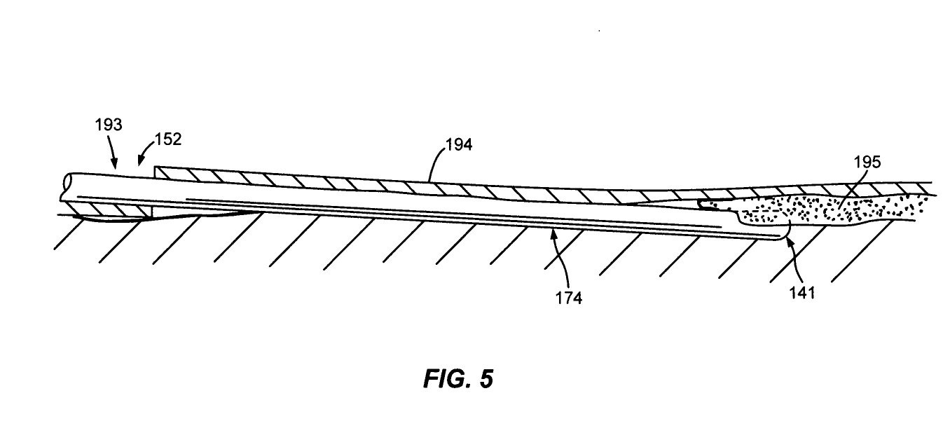

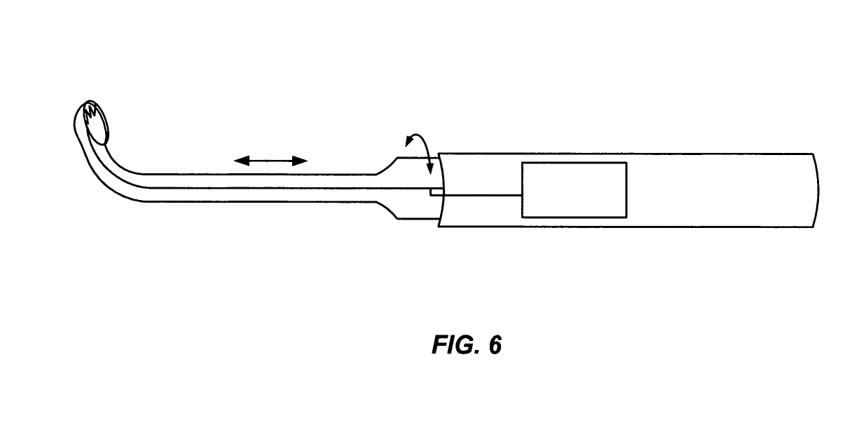

The cutting element 141 is then placed in contact with an undesired vein 195 of the patient as shown in FIGS. 3 and 5. The vacuum suction 160 can be used to draw the vein tissue to be cut into the port 178 of the outer stationary member 170. As the vein is drawn into the port 178, the vein is severed by the cutting element 141 of the reciprocating member 180 and cut into small particles or tiny particles or pieces. The particles of the undesired vein 195 are removed from the surgical site to the vacuum source.

As shown in FIG. 4, the surgeon can move the surgical instrument 120 back and forth under the skin and pushing it in and out in a fan-like or circular fashion as permitted to progressively cut away the vein. The amount of cutting varies with the speed of the cutting element 141, the amount of pressure applied by the surgeon, the sharpness of the cutting edges of the cutting element 141, and the number and size of the ports on the elongated member 180.

After the vein is removed, the incision is closed by conventional techniques, such as, with steri-strips. While suturing of the incision is also possible, it is considered unnecessary due to the small size of the incision, and is also undesirable since suturing the skin can cause scarring. This procedure can then be repeated at another location.

Once the undesired veins are removed, a stocking can be rolled up on the patient's leg and a convatec duoderm elastaplast type stocking can be placed from just proximal to the toes to the upper thigh in order for compression to take place.

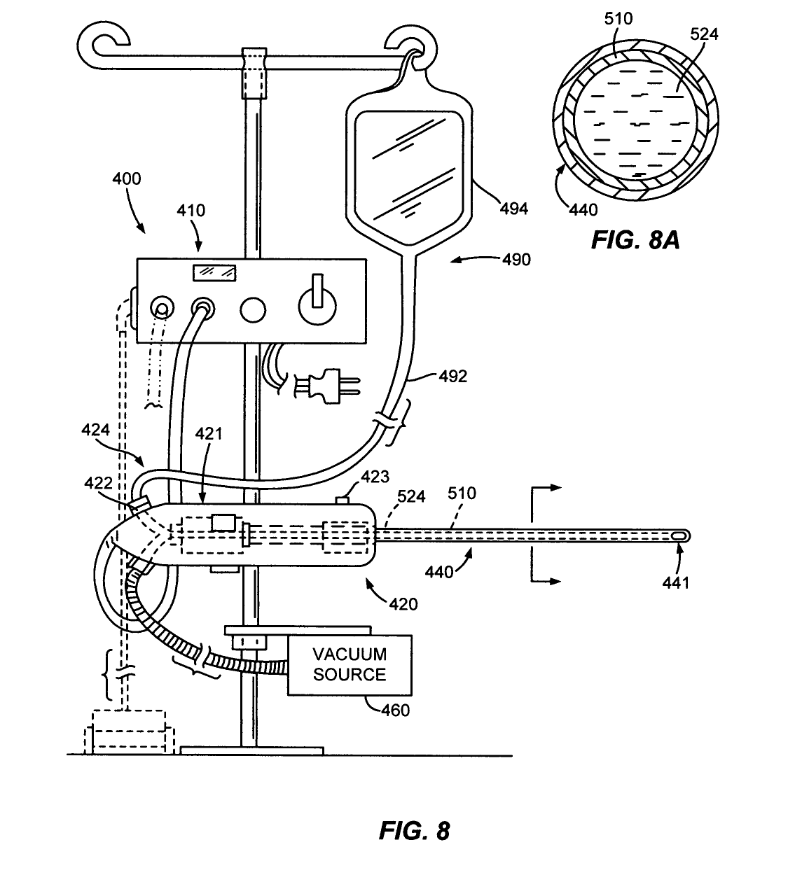

The apparatus and methods of the present invention allow various veins to be removed with minimal scarring and without hospitalization. Moreover, the procedure can be performed on an outpatient basis without any of the usual complications of conventional surgical procedures. The procedure can be performed in a short period of time to avoid physician fatigue, minimize anesthesia time for the patient, and increase the number of procedures possible with a given operating room facility. Referring now to FIG. 8, another surgical system 400 is illustrated which in many respects corresponds in construction and function to the previously described surgical system 100 of FIG. 1. Components of the surgical system 400 which generally correspond to those components of the surgical instrument 100 are designated by like reference numbers in the four-hundred series.

As shown in FIG. 8, the surgical system 400 includes an irrigation assembly 490 that allows saline or other materials to be introduced into the surgical site of the patient. The irrigation assembly 490 preferably includes a hose 492 and a saline bag 494. The hose 492 has an aperture extending therethrough to allow the saline to flow from the saline bag 494 through the hose 492 and into the handpiece assembly 421 via a coupling member 422 when a trigger member 423 is depressed. The saline flows through a conduit 424 of the handpiece assembly and blade assembly and into the surgical site. It is also contemplated that the coupling member 422 can be located at the distal end of the handpiece assembly 421 or on the blade assembly 440. It will be recognized that the tip of the blade assembly 440 can be dipped into saline to introduce saline into the surgical site or the saline can flow through a conduit extending through the elongated member of the blade assembly 440 to the cutting element 441. The saline can be suctioned from the surgical site when the vacuum source 460 is activated.

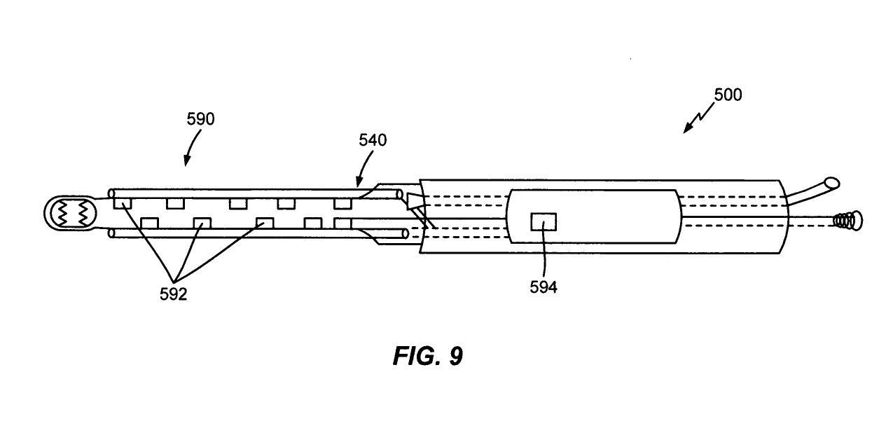

Referring now to FIG. 9, another surgical system 500 is illustrated which in many respects corresponds in construction and function to the previously described surgical system 400 of FIG. 8. Components of the surgical system 500 which generally correspond to those components of the surgical instrument 400 of FIG. 8 are designated by like reference numbers in the five-hundred series.

As shown in FIG. 9, a light source 590 is provided to aid in visualizing a vein during the vein removal procedure. The light source 590 can provide visualization of the veins in the area of surgery to aid the surgeon during the operation. The light source 590 preferably includes a plurality of LEDs 592 mounted on the elongated member of the blade assembly 540. The LEDs 592 are preferably mounted on the outer surface of the blade assembly 540 and can be arranged in any suitable pattern. For example, the LEDs can be mounted in rows encircling the elongated member of the blade or positioned substantially in the direction of the longitudinal axis of the elongated member. The LEDs 592 are electrically coupled to the control system by wires (not shown).

The surgeon can active the LEDs 592 by a switch 594 mounted on the handpiece assembly. The LEDs 592 enable the surgeon to illuminate the area in close proximity to the veins being removed, greatly improving the accuracy, efficacy, and efficiency of the procedure. The LEDs 592 also enable the surgeon to view small, branching veins which can also be advantageously removed using the surgical instrument 500. The blade assembly can be detachably coupled to the handpiece assembly so that it can be disposable of after use.

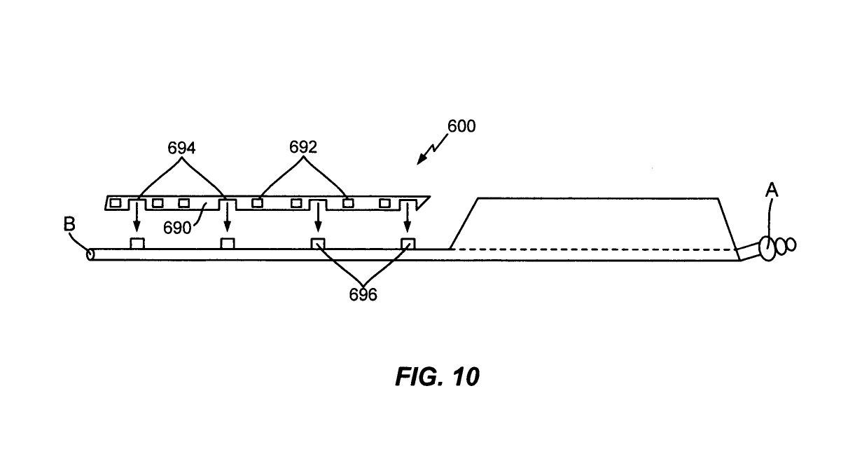

Referring now to FIG. 10, another handpiece assembly 600 is illustrated which in many respects corresponds in construction and function to the previously described handpiece assembly 400 of FIG. 8. Components of the surgical system 600 which generally correspond to those components of the surgical instrument 400 of FIG. Bare designated by like reference numbers in the six-hundred series.

As shown in FIG. 10, a disposable light attachment 690 is shown that mounts to the elongated member of the blade assembly 640. The light attachment 690 preferably includes a plurality of LEDs 692 mounted to the outer surface of the light attachment 690. The light attachment 690 has a plurality of receptacles 694 (four being shown) to detachably couple to a plurality of connectors 696 (four being shown) on the blade assembly 640. It will be recognized that any suitable attachment mechanism can be used to attach the light attachment 690 to the blade assembly 340. The LEDs 692 enables the surgeon to illuminate the area in close proximity to the veins being removed, greatly improving the accuracy, efficacy, and efficiency of the procedure, and enabling the surgeon to view small, branching veins which can also be advantageously removed using surgical instrument 600.

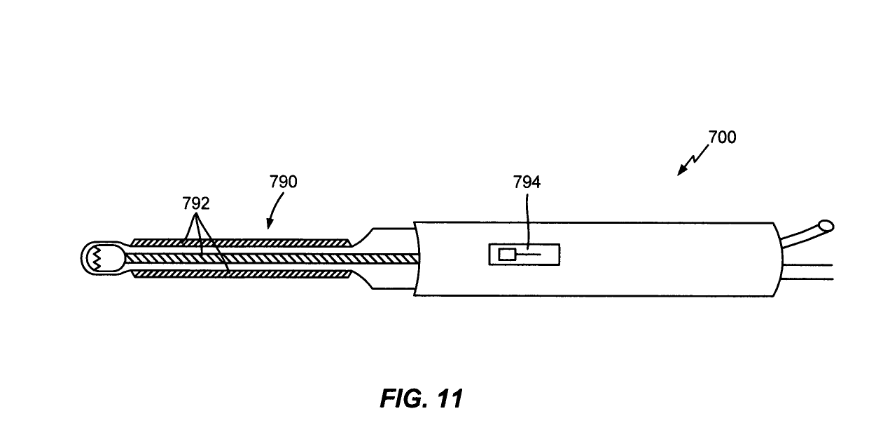

Referring to FIG. 11, another surgical system 700 is illustrated which in many respects corresponds in construction and function to the previously described surgical system 400 of FIG. 8. Components of the surgical system 700 which generally correspond to those components of the surgical instrument 400 of FIG. 8 are designated by like reference numbers in the seven-hundred series.

As shown in FIG. 11, the blade assembly of the surgical device includes a light source 790. The light source 790 can provide visualization of the veins in the area of surgery to aid the surgeon during the operation. The light source 790 he light source 790 can be activated by the trigger mechanism 794.

The light source 790 preferably includes a plurality of fiber optic illuminating devices 792 to aid in visualizing a vein during the cutting procedure. The light source is positioned subcutaneously and in close proximity to the vein to be cut, e.g., about 1 to 2 cm from the vein, with light directed at the vein from below or from the side of the vein causing the vein to be shadowed. This enables the surgeon to see the vein through the patient's skin, greatly improving the accuracy, efficacy, and efficiency of the procedure, and enabling the surgeon to view small, branching veins which can also be advantageously removed using surgical instrument 700.

The fiber optic illuminating devices 792 preferably include fiber optic bundles. The fiber optic bundle are attached to the outer surface of the elongated member and positioned substantially in the direction of a longitudinal axis, A, of elongated member.

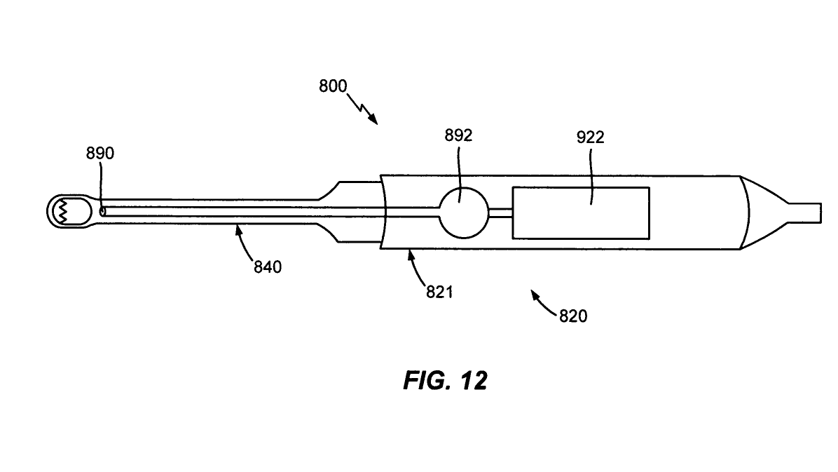

Referring to FIG. 12, another surgical system 800 is illustrated which in many respects corresponds in construction and function to the previously described surgical system 400 of FIG. 8. Components of the surgical system 800 which generally correspond to those components of the surgical instrument 400 of FIG. 8 are designated by like reference numbers in the eight-hundred series.

As shown in FIG. 12, the surgical instrument 820 includes an imaging device. Preferably, the imaging device includes a camera lens 890 and a camera processor 892. The camera lens 890 in positioned near the distal end of the elongated member 840 of the handpiece assembly 821 and the camera processor 892 is disposed within the housing assembly 821. The camera processor is coupled to the control unit (not shown) which can display the images. The imaging device of the surgical instrument 820 allows a surgeon to take still images or video of the vessels, particularly small branches of veins which are otherwise difficult to see.

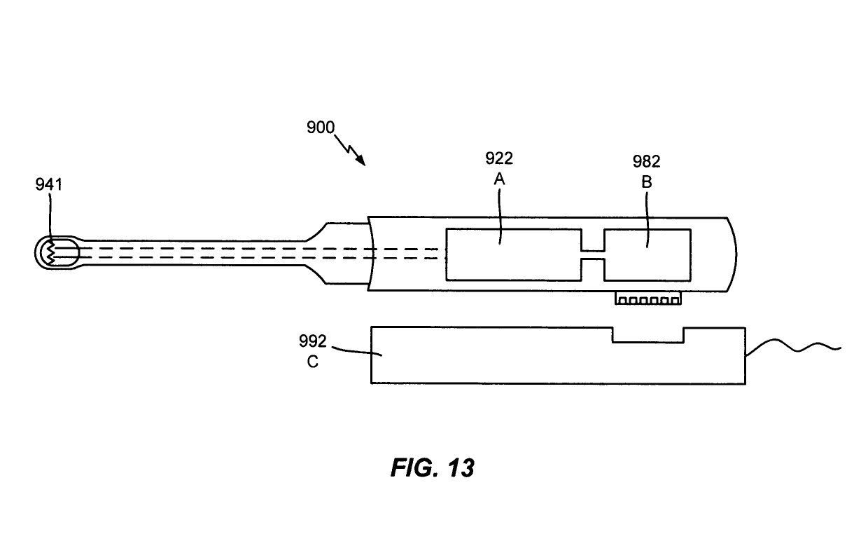

Referring to FIG. 13, another surgical system 900 is illustrated which in many respects corresponds in construction and function to the previously described surgical system 400 of FIG. 8. Components of the surgical system 900 which generally correspond to those components of the surgical instrument 400 of FIG. 8 are designated by like reference numbers in the nine-hundred series.

As shown in FIG. 13, the surgical instrument 920 includes a power source integrated into the housing assembly. The power source 982 provides power to the motor 922 so that the drive shaft can reciprocate the cutting element. Preferably, the power source 982 includes a rechargeable battery. The battery can be charged by attaching the surgical assembly to a charging device 992.

Although the present invention has been described in detail by way of illustration and example, it should be understood that a wide range of changes and modifications can be made to the preferred embodiments described above without departing in any way from the scope and spirit of the invention. Thus, the described embodiments are to be considered in all respects only as illustrative and not restrictive, and the scope of the invention is, therefore, indicated by the appended claims rather than the foregoing description. All changes that come within the meaning and range of equivalency of the claims are to be embraced within their scope.

I claim:

1. A device for removing veins in a venous system of a patient comprising:
an elongated shaft having a first end and a second end, the second end of elongated shaft having an aperture, a cutting edge disposed on the distal edge of the aperture; and
a reciprocating member disposed within the elongated shaft and having a first end and a second end, the second end of the reciprocating member having a cutting member to contact against the cutting edge of the aperture; and the reciprocating member be reciprocated in response to electrical energy.

2. The device of claim 1, further comprising a plurality of light emitting diodes (LEDs) attached to the outer surface of the elongated shaft.

3. The device of claim 1, further comprising a light attachment detachably coupled to the elongated shaft.

4. The device of claim 3, further comprising a plurality of LEDs attached to the light attachment.

5. The device of claim 1, further comprising at least one fiber optic bundle attached to the elongated shaft, wherein the fiber optic bundle is positioned substantially the direction of the longitudinal axis of the elongated shaft.

6. The device of claim 1, further comprising an imaging device that includes a camera lens and a camera processor, wherein the camera lens is positioned near the distal end of the elongated shaft and the camera processor is positioned within a housing of the device.

7. The device of claim 1, wherein the device includes a housing having an integrated power source.

8. The device of claim 1, wherein the device includes a housing having an integrated power source.

9. The device of claim 1, wherein the elongated shaft is curved at a desired angle.

10. A device for cutting tissue comprising:
a surgical instrument having a distal end configured for subcutaneous insertion into a patient's tissue and positioning in proximity of the tissue, the surgical instrument including:
a housing;
a cylindrical outer tube defining a lumen, wherein the cylindrical outer tube extends from the housing; and
a cylindrical inner tube located within the cylindrical outer tube, and having an inner tube aperture extending through the cylindrical inner tube, the cylindrical inner tube being reciprocated within the cylindrical outer tube to cut tissue.

11. The device of claim 10, further comprising a plurality of light emitting diodes (LEDs) attached to the outer surface of the cylindrical outer tube.

12. The device of claim 10, further comprising a light attachment detachably coupled to the cylindrical outer tube.

13. The device of claim 12, further comprising a plurality of LEDs attached to the light attachment.

14. The device of claim 10, further comprising at least one fiber optic bundle attached to the elongated shaft, wherein the fiber optic bundle is positioned substantially the direction of the longitudinal axis of the elongated shaft.
